(19) United States
(12) Patent Application Publication
(10) Pub. No.: US 2024/0062875 A1
(43) Pub. Date: Feb. 22, 2024

(54) HEAD MOUNTED DISPLAY AND BLOOD SUGAR LEVEL MANAGEMENT METHOD EMPLOYED IN SAME

(71) Applicant: Maxell, Ltd., Kyoto (JP)

(72) Inventors: Mayumi NAKADE, Kyoto (JP); Shigeyuki ITOU, Kyoto (JP); Osamu KAWAMAE, Kyoto (JP)

(21) Appl. No.: 18/267,588

(22) PCT Filed: Dec. 17, 2020
(86) PCT No.: PCT/JP2020/047276

§ 371 Date: Jun. 15, 2023

(51) Int. Cl. G16H 20/60; G02B 27/01

(52) U.S. Cl. CPC G16H 20/60; G02B 27/0172; G02B 2027/0138; G02B 2027/014; G02B 2027/0141

(57) ABSTRACT

A head mounted display and a blood sugar level management method are employed to provide effective dietary guidance for suppressing the blood sugar level to within a certain range. In order to achieve the aforementioned purpose, a head mounted display displays AR objects in a real space is provided, wherein: the head mounted display includes a camera that acquires a captured image by capturing an image of the real space, a blood sugar level sensor that measures the blood sugar level, and a control device. The control device determines, from information about a real food obtained from the captured image and information obtained from the blood sugar level sensor, the amount eaten in one bite and the time interval until the next bite is eaten; and the amount eaten in one bite and the time interval until the next bite is eaten are displayed as the AR objects.

### TECHNICAL FIELD

This invention relates to a head mounted display (hereinafter referred to as HMD) for use in a mixed reality (MR: Mixed Reality) system that superimposes a real space and a virtual space (also referred to as virtual object).

### BACKGROUND ART

To prevent obesity and diabetes, it is effective to keep blood sugar level within normal levels by preventing rapid increases in blood sugar level. In order to keep blood sugar level within the normal range, it is important to pay attention to the order in which dish is eaten and to prevent overeating and eating too quickly during meals. Conventionally, regarding meal control, it is known to monitor and control the number of times of mastication and heart rate.

Patent Document 1 is a background technology in this technical field. Patent document 1 describes a point where the system recognizes dishes and eating behaviors such as bite size and the number of times of mastication from camera images, dynamically creates individual criteria for bite size and the number of times of mastication from standard bite size for each dish, and estimates and outputs issues based on comparison between the created criteria and the actual bite size.

### CITATION LIST

### Patent Document

Patent Document 1: JP 2018-33624 A

### SUMMARY OF THE INVENTION

### Problems to be Solved by the Invention

In Patent Document 1, it was not possible to correlate the amount of actual eaten with the real-time rise in blood sugar level in relation to the amount eaten, making it impossible to provide optimal dietary guidance to keep blood sugar level within a certain range.

Here, a HMD is a device that is worn on the head and displays images on a glasses-type or goggles-type display. This device is equipped with a camera, a plurality of sensors such as a sensor for measuring the distance to an object and a GPS sensor for measuring the position, a CPU for image processing, a battery, and the like. On the other hand, non-invasive blood sugar sensors have been developed that can measure blood sugar level.

Therefore, a method of effectively managing fluctuations in blood sugar levels during a meal using a HMD and a blood sugar level sensor is conceivable.

In view of the above, the present invention, by utilizing a HMD and a blood sugar sensor, aims to provide a HMD and a method of controlling blood sugar level by presenting specific eating methods in real time, such as controlling eating time to avoid eating too fast and recommending the order in which dish is eaten, and suppressing rapid rise in blood sugar level.

### Solutions to Problems

This invention, to cite one example, is a HMD that displays AR objects in real space, wherein: the head mounted display includes a camera that acquires a captured image by capturing an image of the real space, a blood sugar level sensor that measures the blood sugar level, and a control device; the control device determines, from information about a real food obtained from the captured image and information obtained from the blood sugar level sensor, the amount eaten in one bite and the time interval until the next bite; and the amount eaten in one bite and the time interval until the next bite are displayed as the AR objects.

### Effects of the Invention

According to the present invention, it is possible to provide a HMD and blood sugar level management method employed in same that can provide dietary guidance effective in keeping blood sugar level within a certain range.

### BRIEF DESCRIPTION OF THE DRAWINGS

FIG. 1 is an external image diagram of a HMD in a first embodiment.

FIG. 2 is an example of a guide display for an eating method in the first embodiment.

FIG. 3 is a display example of a recommended amount of one bite for dish that the wearer of HMD is about to eat in the first embodiment.

FIG. 4A to FIG. 4E are display examples of the number of times of mastication in the first embodiment.

FIG. 5 is an example of displaying a dish recommended to be eaten next in the first embodiment.

FIG. 6A and FIG. 6B are another examples of a guide display for an eating method in the first embodiment.

FIG. 7 is a diagram simulating the relationship between the blood sugar level and the time required for a meal in the first embodiment.

FIG. 8A to FIG. 8E are another example of a guide display for an eating method in the first embodiment.

FIG. 9 is a diagram illustrating cooperation between the HMD and a mobile terminal in the first embodiment.

FIG. 10 is a hardware configuration diagram of the HMD in the first embodiment.

FIG. 11 is a processing flowchart for initial setting of the blood sugar level management method in the first embodiment.

FIG. 12 is a processing flowchart of the blood sugar level management method in the first embodiment.

FIG. 13 is an another example of the processing flowchart of the blood sugar level management method in the first embodiment.

FIG. 14 is a processing flowchart of abnormal blood sugar level processing in the first embodiment.

FIG. 15 is a processing flowchart of blood sugar level progress observation processing in the first embodiment.

FIG. 16 is a system configuration diagram for realizing the blood sugar level management method in a second embodiment.

FIG. 17 is an example of a database of carbohydrate and fiber amounts for each restaurant menu in the second embodiment.

FIG. 18A to FIG. 18D are examples of display in case of hypoglycemia in a third embodiment.

FIG. 19 is a processing flowchart of the process corresponding to hypoglycemia in the third embodiment.

FIG. 20 is a system configuration diagram for explaining emergency contact in case of hypoglycemia in the third embodiment.

FIG. 21A to FIG. 21C are explanatory diagrams of a display to make an amount for one bite appear larger in a fourth embodiment.

### MODE FOR CARRYING OUT THE INVENTION

Hereinafter, embodiments of the present invention will be described with drawings.

### First Embodiment

In the present embodiment, an eating method that uses the HMD 100 and makes it possible to suppress an increase in the blood sugar level within a certain range during meals will be described.

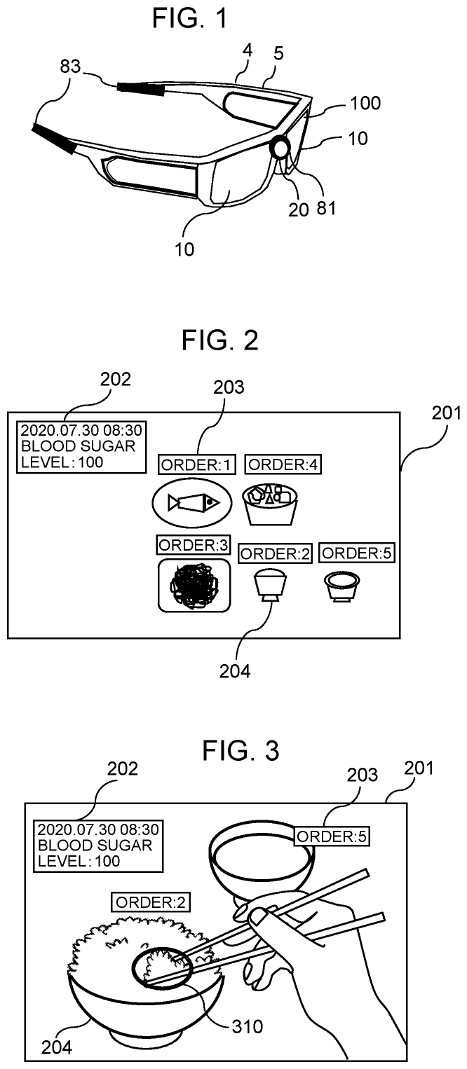

FIG. 1 is an external image diagram of a HMD used in this embodiment. As shown in FIG. 1, the HMD 100 has a display 10, a camera 20, a microphone 81, various sensors 5, such as a distance sensor that measures distance, which is the position of an object captured by the camera section, an acceleration sensor, which is a motion detection sensor that measures movement such as vibration and acceleration of the HMD, a gyro sensor that measures rotation, a blood sugar level sensor, a speaker 83, a battery 9 (not shown), and a control circuit (control device) 4. Note that the display 10 is a transparent or semi-transparent display, and the wearer of the HMD 100 can see virtual objects and images superimposed on the outside scenery (augmented reality, hereinafter referred to as AR) displayed on the display 10.

Next, a display example of the HMD 100 that guides an eating method for suppressing an increase in blood sugar level within a certain range according to the present embodiment will be described.

FIG. 2 is an example of a guide display for an eating method in this embodiment. In FIG. 2, 201 is a display area of the display 10 of the HMD 100, 202 and 203 are AR objects, and 204 is the actual dish.

AR object 202 is the result of measuring the blood sugar level of the person wearing the HMD100 with a blood sugar level sensor. AR object 203 indicates the best order of dishes to eat from the viewpoint of controlling the rise in blood sugar level by capturing the entire dish with the camera 20, detecting the classification of the dish and ingredients from the image data and database, and estimating the amount of sugar and ease of absorption of sugar for each dish from the sugar content data for each dish. The wearer can determine the order in which to eat the dishes by referring to these indications. The displayed order of eating is not mandatory, but only for reference. In FIG. 2, the entire dish is within the field of view, but it is not necessary to capture the entire dish at once with the camera 20. It is also possible to estimate the dishes sequentially detected from the moving images captured by the camera 20, and display the order of eating of only the dishes detected from the sugar content data of the detected dishes. In this case, for example, the wearer can find the numbered dishes in the eating order by looking over the entire dish.

The amount of each dish may be measured from three-dimensional datameasured by distance sensors or camera image processing, and the sugar content of each dish may be calculated together with the sugar content data of the dishes and ingredients to determine and display the amount of dishes and dish that may be eaten. In this case, for example, dishes that are not recommended to be eaten are not assigned a number and are indicated as not recommended. The recommended amount to eat may also be displayed along with the order of eating. The recommended amount to eat may be displayed numerically, for example, or an AR object indicating the recommended amount to eat or the area not to eat may be overlaid on the dish.

FIG. 3 is a display example of a recommended amount of one bite for dish that the wearer of HMD 100 is about to eat in this embodiment. In FIG. 3, the same functions as in FIG. 2 are marked with the same symbols, and their descriptions are omitted. In FIG. 3, 310 is an AR object, indicating the amount of one bite. In order to keep the rise in blood sugar level within a certain range, the amount of one bite of dish is also important to prevent eating too quickly. Therefore, the recommended amount, for example, a circle mark as shown in AR object 310 is displayed in AR to provide guidance on the amount to be taken with chopsticks, a spoon, or the like. This amount of one bite guidance is displayed when the chopsticks, a spoon, or the like comes close to the dish to be eaten. Although the AR object 310 is indicated by a circle here, it is possible to make the wearer recognize the amount to be eaten more easily by displaying it in 3D according to the actual dish. In addition, the amount of one bite to be displayed is changed according to the dish and the amount of change in the wearer's blood sugar level.

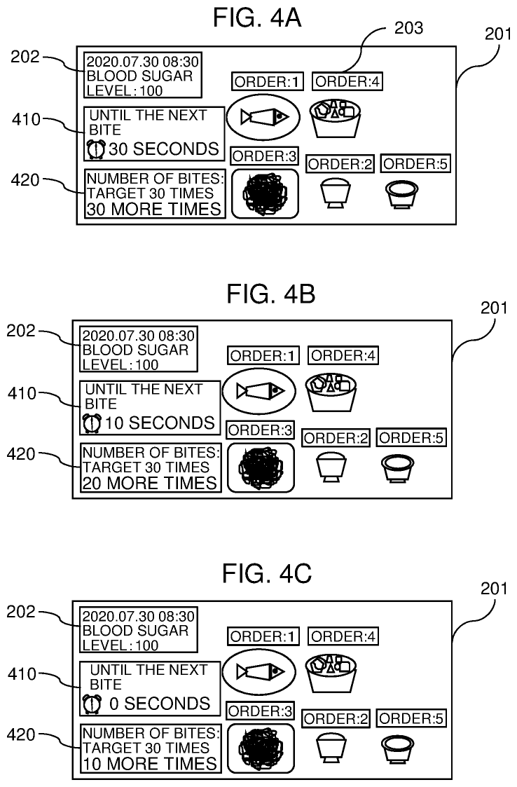

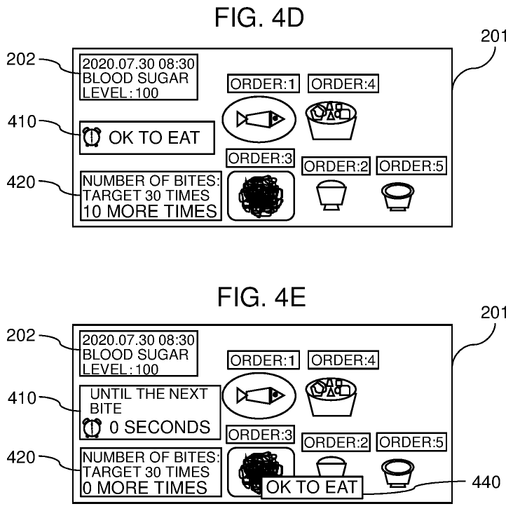

FIG. 4A to FIG. 4E are display examples of the number of times of mastication in this embodiment. In FIG. 4A to FIG. 4E, the same functions as in FIG. 2 are marked with the same symbols, and their explanations are omitted. In FIG. 4A to FIG. 4E, FIG. 4A shows the display immediately after taking a bite. In FIG. 4A, 410 is an AR object that displays the time interval until the next bite. 420 is an AR object that displays the number of times of mastication, and displays the number of times of mastication as a reference and the remaining number of times of mastication from the reference number of times of mastication after the start of counting the actual number of times of mastication. For example, in 410, the time interval until the next bite is displayed as 30 seconds, and in 420, the reference number of times of mastication is displayed as 30 times and the remaining number of times of mastication is displayed as 30 more times.

FIG. 4B shows the display in the state 20 seconds after taking a bite. In 410, the time interval until the next bite is displayed as 10 seconds, and if 10 mastication times are detected in 20 seconds, the remaining number of times of mastication is displayed as 20 more times in 420 as a message to encourage the user to increase the number of times of mastication. Note that the numerical values in 410 and 420 do not transition from FIG. 4A to FIG. 4B, but the display changes every second in 410 and every mastication in 420, for example.

FIG. 4C shows the display in the state 30 seconds after taking a bite. 410 shows the time interval until the next bite is taken as 0 seconds left, and if 20 mastication times are detected in 30 seconds, the remaining number of times of mastication is displayed as 10 more times in 420.

FIG. 4D is an example of judging whether to display that it is okay to eat only after the passage of time. As in FIG. 4C, FIG. 4D shows the display in the state 30 seconds after taking a bite. FIG. 4D is a case in which only the passage of time is used as a criterion, and when 30 seconds, the predetermined time interval before the next bite, elapses, AR object 430 is displayed indicating that it is OK to eat. Note that as in Fig. FIG. 4C, and if 20 mastication times are detected in 30 seconds, the remaining number of times of mastication is displayed as 10 more times in 420.

FIG. 4E is an example of judging whether to display that it is okay to eat based on the passage of time and the number of times of mastication. As in FIG. 4C, FIG. 4E shows the display in the state 30 seconds after taking a bite. In addition, this is the state in which the number of times of mastication is detected to be 30 times or more. In this case, AR object 440 is displayed indicating that it is OK to eat. In 410, the time interval until the next bite is taken is 0 seconds left is displayed, and in 420, the remaining number of times of mastication is displayed as 0 times left. Note that 410 and 420 may not be displayed. In addition, when the number of times of mastication per mouth satisfies the reference number of times of mastication, a display indicating that the number of times of mastication is appropriate may be displayed in the display area 201, for example, a message such as “Please continue eating at that tempo” or a smiling icon may be displayed.

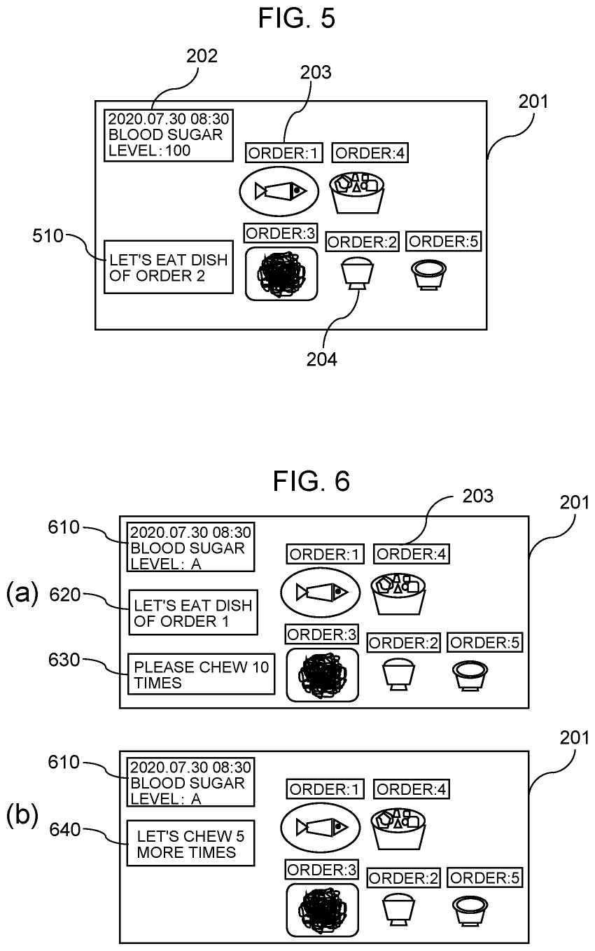

FIG. 5 is an example of displaying a dish recommended to be eaten next in this embodiment. In FIG. 5, the same functions as in FIG. 2 are marked with the same symbols, and their descriptions are omitted. In FIG. 5, 510 is an AR object display of the order in which to eat next. The order of eating is not mandatory, but the order of eating is recommended. If the wearer is forgetful, teaching the current action will make it easier for the wearer to take more appropriate action.

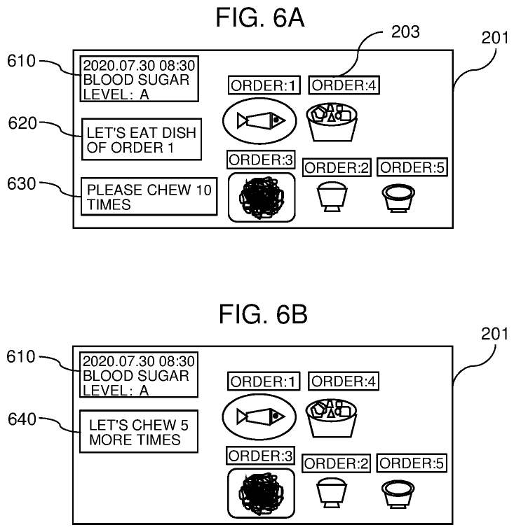

FIG. 6A and FIG. 6B are another examples of a guide display for an eating method in this embodiment. In FIG. 6A and FIG. 6B, the same functions as in FIG. 2 are marked with the same symbols, and their descriptions are omitted. In FIG. 6A and FIG. 6B, FIG. 6A is a standard example at the start of a meal in which the blood sugar level detected by the blood sugar level sensor at the start of the meal, the recommended cooking order to keep the increase in blood sugar level within a certain range, and the standard number of times of mastication when eating that dish are displayed in AR. AR object 610 is the blood sugar level detected by the blood sugar level sensor, AR object 620 is the recommended cooking order, and AR object 630 is an example of displaying the standard number of times of mastication. The measured blood sugar level may be displayed as it is in numerical values as shown in FIG. 2, but for example, as shown in AR object 610, the normal value range is displayed as A, below the normal value range as B, and above the normal value range as C, etc. In addition, guidance on the recommended amount for one mouthful is also displayed as described in FIG. 3.

FIG. 6B is an example of displaying a message related to mastication, when eating dish, count the number of times of mastication, and AR object 640 displays “Let's chew 5 more times” so as to masticate until the count reaches the reference count.

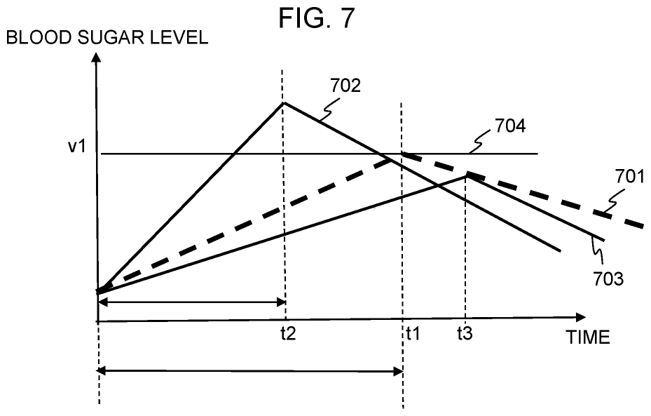

Here is a conceptual explanation of the relationship between the increase in blood sugar level and the time required for a meal. FIG. 7 is a diagram simulating the relationship between the blood sugar level and the time required for a meal. The vertical axis shows the blood sugar level and the horizontal axis shows the time from the start of the meal. 701 is a graph of the change in blood sugar level when eating at standard speed, 702 is a graph of the change in blood sugar level when eating quickly, 703 is a graph of the change in blood sugar level when eating slowly, and 704 indicates the maximum blood sugar level v1 when eating at standard speed.

In general, blood sugar level peakabout 60 minutes after a meal and return to pre-meal blood sugar level in about 120 minutes. However, for so-called fast eaters, who take much faster than average time to eat, the amount of carbohydrates and proteins that raise blood sugar level in a short period of time is greater than for people with average meal times, resulting in a steeper curve for the rise in blood sugar level and consequently a greater peak blood sugar value. For example, if a person eats at a standard speed, the blood sugar level reaches the maximum blood sugar level v1 at time t1, as shown in Graph 701. However, in the case of a fast eater who finishes a meal in about 10 minutes, the blood sugar level is at its maximum at time t2<t1, and that value is greater than the maximum blood sugar level v1. Conversely, if the person eats more slowly than the norm, the blood sugar value is maximal at time t3>t1, and that value is smaller than the maximal blood sugar value v1.

For people who have developed diabetes or are one step ahead of developing diabetes, blood sugar level above a certain range are dangerous, as they can worsen symptoms or even lead to the onset of the disease. Furthermore, it is believed that a small increase in blood sugar level is better for weight loss. Therefore, one solution to keep blood sugar level within a certain range is to have adequate meal times. It is also important to decrease the amount to eat.

As one of the measures to slow down the meal time and reduce the amount of meals, it is said to be effective to control the number of times of mastication, that is, to keep the number of times of mastication above a certain number of times (hereinafter referred to as “standard number of times”).

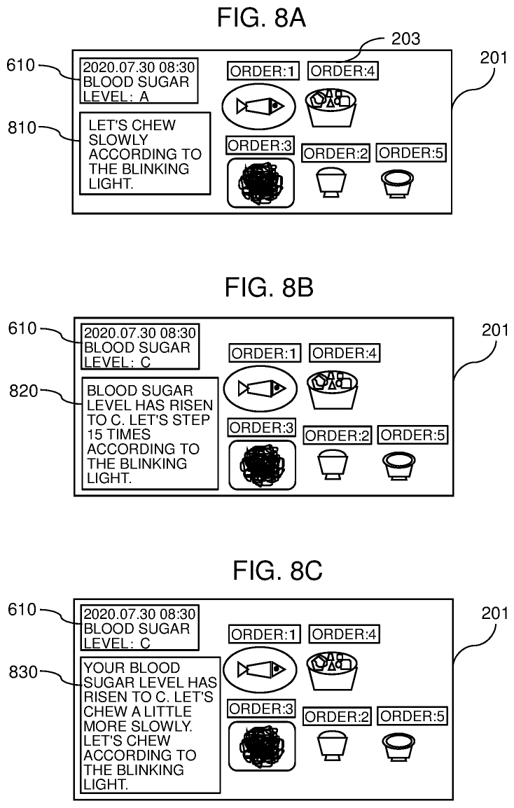

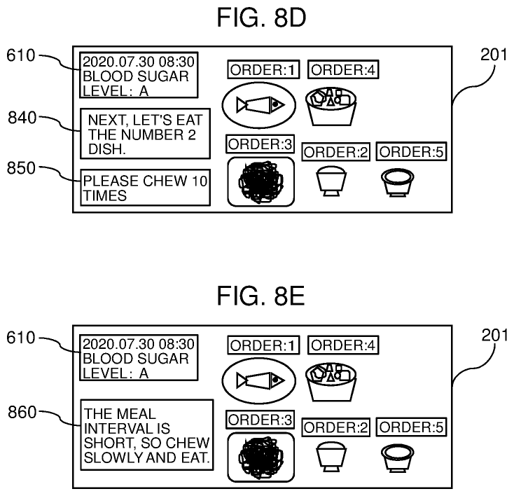

FIG. 8A to FIG. 8E are another examples of a guide display for an eating method in this embodiment. In FIG. 8A to FIG. 8E, the same functions as in FIG. 2 and FIG. 6A and 6Bare marked with the same symbols, and their explanations are omitted. In FIG. 8A to FIG. 8E, in FIG. 8A, when the mastication speed is measured with the HMD 100 and the mastication speed is high, in order to slow down the mastication speed, the display of the HMD 100 displays, for example, blinking light to control the mastication speed, and also displays a message to masticate slowly accordance to the blinking light as indicated by the AR object 810. By increasing the number of times of mastication and lengthening the interval between meals, as shown in FIG. 7, it is possible to moderate the rise in blood sugar level caused by fast eating and to keep it within a certain range.

FIG. 8B shows that if the blood sugar level measured during a meal is determined to be above a certain range, as shown in AR object 610, C is displayed to indicate that the blood sugar level is above the normal range, as well as displaying that the blood sugar level is above the normal range, as shown in AR object 820. Furthermore, if the increase in blood sugar level is judged to be difficult to suppress by increasing the number of times of mastication, etc., since digestion of blood sugar through light exercise such as stepping is effective in lowering blood sugar level, a message such as “Blood sugar level has risen to C. Let's step 15 times according to the blinking light.” is displayed. Whether or not the stepping has been performed is ascertained by the acceleration sensor and other sensors of the HMD 100, and if it has not been performed, a message urging the user to perform the stepping again or the like is displayed. Furthermore, if it is determined that the increase in blood sugar level cannot be suppressed, a message may be displayed urging the user to stop eating.

FIG. 8C shows that when the blood sugar level measured during a meal reaches or exceeds a certain range, but it can be estimated that the blood sugar level will not exceed significantly from the the measured blood sugar level curve, C is displayed to indicate that the blood sugar level is above the normal range as the value of blood sugar level in AR object 610. However, in order to judge that the condition is not so bad that stepping should be implemented, and to instruct the user to eat more slowly, the message “Your blood sugar level has risen to C. Let's chew a little more slowly. Let's chew according to the blinking light.” is displayed as the AR object 830. Furthermore, if the blood sugar level measured during the meal rises above a certain range, for example, the rise in blood sugar level may be suppressed by reducing the recommended bite size, such as by making the display of the AR object 310 in FIG. 3 smaller.

FIG. 8D shows the AR display of the recommended order of dishes to be eaten in effectively controlling the rise in blood sugar level. In order to have them eat the dishes in order, the AR object 840 displays what it wants them to eat next. For example, the AR object 840 displays “Next, let's eat the number 2 dish.”. In addition, the reference number of times of mastication is also displayed by the AR object 850.

FIG. 8E is a display example when, for example, the time between eating breakfast and eating lunch is about one hour earlier than usual. As AR object 860, a message such as “The meal interval is short, so chew slowly and eat.” is displayed. If the time to take the next meal is shortened, it is conceivable that the blood sugar level is not sufficiently lowered. Therefore, it is required to eat more slowly and increase the number of times of mastication.

As described above, in this embodiment, based on the blood sugar level obtained from the sensor data of the HMD 100, the number of times of mastication, the speed of mastication, the meal menu from the camera image, etc., instructions are given in real time on how to eat to keep the rise in the blood sugar level within a certain range.

Note that various displays other than the displays exemplified above are conceivable. For example, a variation is to measure blood sugar level at regular intervals against the blood sugar level at the start of a meal, and to display that blood sugar level show abnormal values only when the values exceed a certain range.

In addition, although the recommended order for eating is displayed when eating a meal, the meal is not always eaten in order. Therefore, the recommended order is indicated each time, or the dish that is desired to be eaten next is displayed larger than the other dishes in an AR display.

In addition, in the above description of the present embodiment, it is assumed that the number of times of mastication, the speed of mastication, etc. are displayed on the AR display as a method of eating to keep the rise in blood sugar level within a certain range, but instructions may be given by voice.

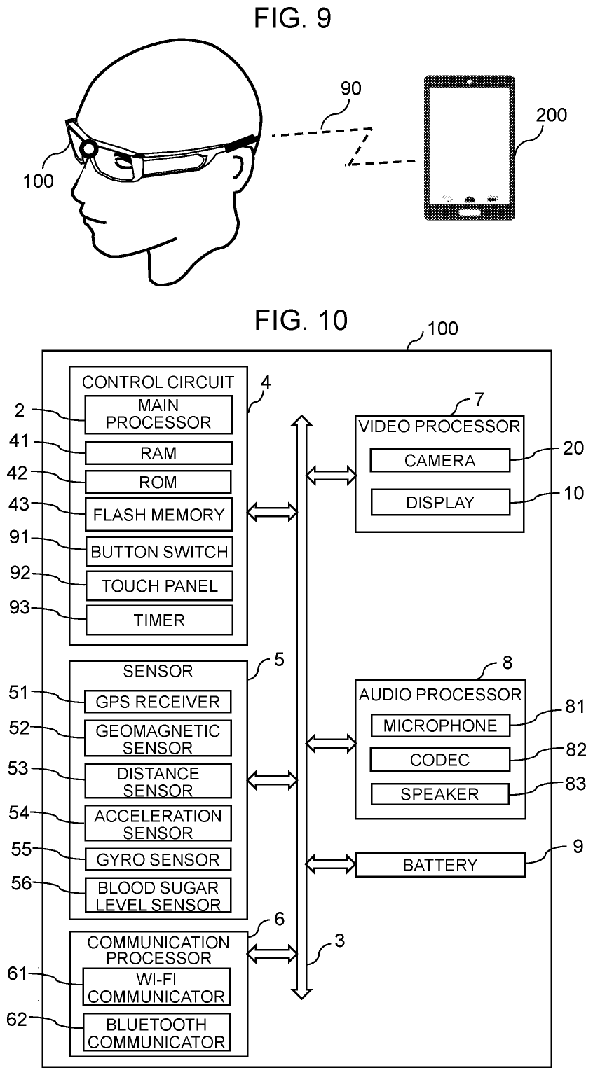

In this embodiment, based on the blood sugar level, meal menu, number of times of mastication, and speed of mastication, etc. collected by the various sensors 5, camera 20, and microphone 81 of the HMD 100, in order to keep the rise in blood sugar level within a certain range, advanced processing instantly judges whether mastication is being performed properly and determines actions to be taken, such as instructions to increase the number of times of mastication or instructions to encourage light exercise, and displays the instructions on a display 10 in an AR. It is assumed that it is difficult to implement these actions using only the HMD 100 with the processing speed of the control circuitry of the HMD 100. In such a case, as shown in FIG. 9, it is possible by sharing tasks such as HMD 100 and mobile terminal (smartphone) 200 can be linked via data communication 90, the advanced processing is performed by mobile terminal 200, and HMD 100 collecting data and displaying the results of the advanced processing by the mobile terminal 200 on the display 10. In addition, some text and other information may be displayed on the mobile terminal 200.

FIG. 10 is a hardware configuration diagram of the HMD in this embodiment. In FIG. 10, the HMD 100 has a control circuit (control device) 4, a sensor 5, a communication processor 6, a video processor 7, an audio processor 8, and a battery 9, which are connected by a system bus 3.

Control circuit 4 has main processor 2, RAM 41, ROM 42, flash memory 43, button switch 91, touch panel 92, and timer 93. Sensor 5 has a GPS (Global Positioning System) receiver 51, a geomagnetic sensor 52, a distance sensor 53, an acceleration sensor 54, a gyro sensor 55, and a blood sugar level sensor 56. Communication processor 6 has a Wi-Fi (registered trademark) communicator 61 and a BlueTooth (registered trademark) communicator 62. Video processing unit 7 has camera (for outside and inside) 20 and display 10. Audio processing device 8 has microphone 81, codec 82, and speaker 83.

The main processor 2 is a so called CPU (central processing unit) or MPU (numerical processing unit) that reads operating programs and information that realize predetermined functions from ROM 42 and flash memory 43 and performs predetermined processing by software processing to control the entire HMD 100.

The system bus 3 is a data communication path that interconnects the main processor 2 and each component in the HMD 100. The main processor 2 and each component in the HMD 100 send and receive various commands and data via the system bus 3.

RAM 41 constitutes a rewritable program work area, such as a work area used by the main processor 2 to execute various programs.

ROM 42 and flash memory 43 store various programs to realize the functions of HMD 100, operation set value, sensor information including detection values from sensors as described below, and various display data such as virtual objects and contents. ROM 42 and flash memory 43 are so-called nonvolatile storage devices that retain the stored information even when power is not supplied to the HMD 100 from the outside.

Flash memory 43 stores operating programs downloaded from the network and various data created by said operating programs. Each operating program stored in flash memory 43 can be updated and extended by downloading from each server device on the network. Furthermore, the flash memory 43 can store contents such as video, still images, and sound downloaded from the network. It can also store data such as video and still images taken by the camera 20.

RAM 41, ROM 42, and flash memory 43 are examples of storage, and other devices, such as semiconductor device memory such as SSD (Solid State Drive), magnetic disk drives such as HDD (Hard Disc Drive), and the like may be used.

The main processor 2 acquires the sensor information for each of the GPS receiver 51, geomagnetic sensor 52, distance sensor 53, acceleration sensor 54, and gyro sensor 55 and blood sugar level sensor 56. The timer 93 also acquires time measurements associated with each event, such as mastication speed and time interval of possession. Using various sensor information, the main processor 2 acquires the number of times of mastication and mastication speed, grasps the state of exercise such as stepping, calculates the blood sugar level, acquires the distance data to the object acquired by the camera, and furthermore, detects the state of the HMD 100 such as position, tilt, orientation, motion, etc. Also, the HMD 100 may be further equipped with other sensors such as an illuminance sensor, a proximity sensor, and an altitude sensor.

The Wi-Fi communicator 61 and BlueTooth communicator 62 transmit and receive data with the mobile terminal 200 via wireless communication, and also transmit and receive data with each server on the network by connecting to a network such as the Internet via a wireless access point.

Although not shown in the figure, the communication processor 6 may have a telephone network communication function, such as GSM (registered trademark) (Global System for Mobile Communications), W-CDMA (Wideband Code Division Multiple Access), CDMA2000, UMTS (Universal Mobile Telecommunications System), and other third generation mobile communication systems (hereinafter referred to as “3G”), alternatively, it may have a communication system called LTE (Long Term Evolution) system, fourth generation (4G), or fifth generation (5G). With this telephone network communication function, it is possible to connect to a communication network through a base station using a mobile communication network and to transmit and receive information to and from a server on the communication network. Note that the wireless communication function and the telephone network communication function are equipped with encoding and decoding circuits, antennas, and the like for their functions, respectively, for the functions. The HMD 100 may also be equipped with other communication I/F, such as infrared communication I/F.

The camera 20 has a function to photograph the outside of the HMD 100 (out-camera function in a mobile terminal (smartphone)). The out-camera function is used to photograph the entire meal, each dish, the spoon and chopsticks used for the meal, and the amount of dish actually scooped with the spoon, etc. (amount of dish in mouth). The camera 20 is a camera that inputs image data of surroundings or objects by converting light input from a lens into an electrical signal using an electronic device such as a CCD (Charge CCD) or CMOS (Complementary Metal Oxide Semiconductor) sensor.

The display 10 is, for example, a display device such as a liquid crystal panel, and provides image data to the wearer of the HMD 100. The HMD 100 includes a video RAM (not shown), and virtual objects, images, text, etc. are displayed in AR on the screen of the display 10 based on the image data input to the video RAM. Note that the display 10 is transparent or semi-transparent.

The microphone 81 converts the voice of the wearer of the HMD 100 and surrounding sounds, etc. into audio data to be input. The speaker 83 outputs audio information and the like. The codec 82 performs encoding and decoding processing of encoded audio signals as necessary.

The button switch 91 and touch panel 92 are operation devices for inputting operation instructions to HMD 100. The operation devices are not limited to the button switch 91 and touch panel 92. For example, a separate portable terminal device (e.g., smartphone or tablet device) connected via wired or wireless communication may transmit operation signals for the HMD 100, and the HMD 100 may receive the operation signals and operate according to these operation signals. Voice may also be input from the microphone 81, and the main processor 2 may perform voice recognition processing to generate an operation signal and control the operation of the HMD 100. Also, the blood sugar level sensor 56 may be installed in a wristwatch-type device that is separate from the HMD 100.

In addition, the example configuration of HMD 100 shown in FIG. 10 includes some configurations that are not essential for this embodiment, but the effect of the present embodiment is not affected even with a configuration in which these are not provided. Further configurations not shown in the figure, such as a digital broadcast receiving function and an electronic money payment function, may be added.

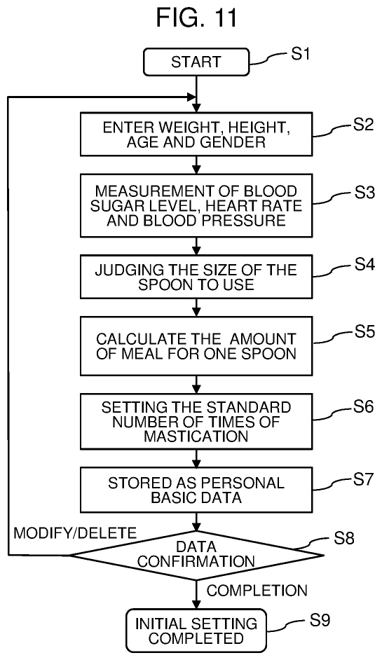

FIG. 11 is a processing flowchart for initial setting of the blood sugar level management method for instructing the eating method, such as the number of times of mastication and the time interval until the next bite, to keep the rise in blood sugar level within a certain range in this embodiment. In FIG. 11, first, in order to control the rise of blood sugar level, it is necessary to initialize basic information such as the amount of dish to be eaten and physical data of the eater. To this end, in step S1, the user of this system wears the HMD 100 and selects the start of the initial setting with button switch 91. Next, in step S2, the weight, height, age, gender, etc. of the target person (the wearer of the HMD 100) who needs to control the rise in blood sugar level within a certain range during meals is selected and input on the touch panel 92. Then, in step S3, the blood sugar level sensor 56 of the sensor 5 of the HMD 100 and the like are used to measure the blood sugar level, heart rate, and blood pressure.

Next, in step S4, the size of the spoon used for the meal is measured by photographing the spoon with the camera 20. In step S5, the amount of meal for one spoon is calculated. Then, in step S6, a general number of times of mastication is set based on the data entered in step S2. Then, in step S7, the data from steps S2 to S6 are stored in the flash memory 43 with an identification name as the personal basic data of the HMD 100 wearer.

In step S8, the input data is confirmed, and if there is no change or error, the process proceeds to step S9 for completing the initial setting, and the data in the flash memory is fixed. If there are any changes or errors, return to step S2 and start the entire initial setup again. Note that only the relevant items may be corrected. In addition, although the above describes the case of using a spoon, it goes without saying that other items such as chopsticks or forks may also be used.

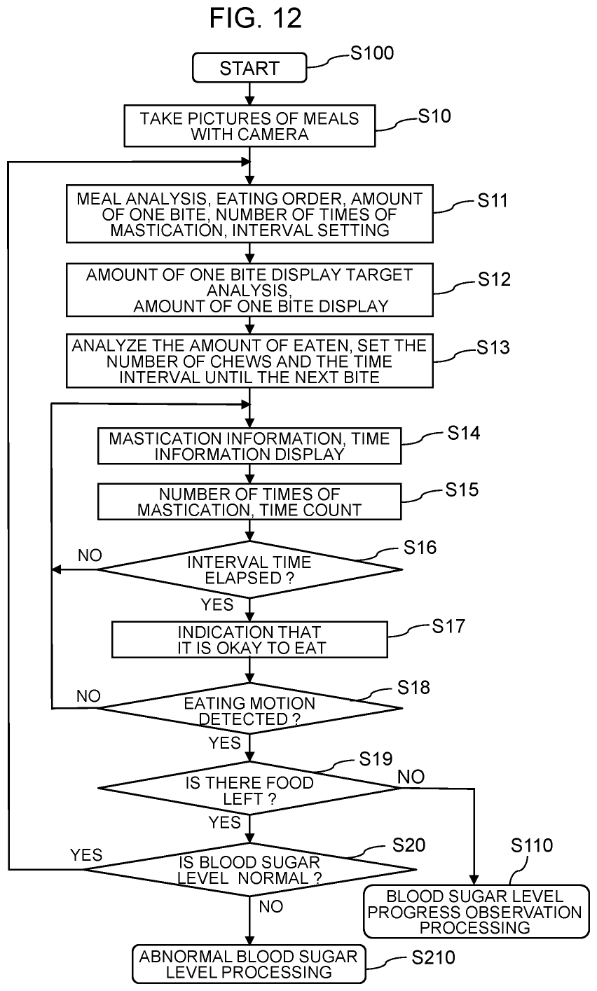

FIG. 12 is a processing flowchart of the blood sugar level management method that provides instructions for controlling normal blood sugar level, such as the number of times of mastication in this embodiment. In FIG. 12, first, in step S100, the user of this system wears the HMD 100 and selects the start of a meal with the button switch 91. Let us assume that the example is a meal at home and all the dishes for the meal are on the table. Next, in step S10, the entire meal is photographed by the camera 20 to grasp what dishes are present. Next, in step S11, the type of dish is roughly determined from the photographed dish, the amounts of carbohydrates, fibers, etc. in the dish related to the blood sugar level are estimated, and the order of the dish to be eaten is determined so as to slow the rise in the blood sugar level. In addition, from the personal data stored in flash memory 43, the spoon to be used, the amount of dish for one bite, the standard number of times of mastication, and the standard time interval until the next bite and so on are set in the program for controlling the increase in the blood sugar level.

Next, in step S12, when the meal is started, the recommended amount to put on the spoon is displayed on the dish in AR (see FIG. 3), and the actual amount is ascertained. Then, in step S13, the amount of actually eaten is ascertained from the amount placed on the spoon and the amount remaining on the spoon, and the recommended number of times of mastication and the time interval until the next bite are analyzed and set. Then, in step S14, the number of times of mastication and the time interval until the next bite analyzed and set in S13 are displayed in AR on the display 10.

Next, in step S15, the actual number of times of mastication and the time until the next bite is eaten are counted using the various sensors 5 of the HMD 100, and these are displayed (see FIG. 4C). Then, in step S16, it judges whether the interval time until the next bite, which is the standard, has passed, and if not, it returns to S14 to display the remaining number of times of mastication and time information. If the interval time until the next bite, which is the standard, has passed, the system moves to step S17 and a message indicating that it is OK to eat is displayed (see FIG. 4D).

Then, in step S18, it determines whether the user's eating motion is detected, and if not, it returns to S14 to continue displaying and counting the time and the number of times of mastication until the user enters the next bite motion. When the user's eating motion is detected, the process proceeds to step S19 to check the remaining food. If there is still food left, move to step S20 to measure the blood sugar level with the blood sugar level sensor of the HMD 100 and check whether the elevated value is within a certain range (normal range). If normal, return to S11 and repeat the same procedure.

If there are no dishes left in step S19, the process proceeds to a blood sugar level progress observation processing for observing how the previously measured blood sugar level in S20 fluctuates (S110). Further, when the increased value of the blood sugar level exceeds a certain range (normal range) in step S20, it is determined that the blood sugar level is abnormal, and the process proceeds to abnormal blood sugar level processing (S210).

In the above flow, even when the interval time until the next bite has elapsed, the display and counting of the time and the number of times of mastication until the user enters the next bite motion, but the display and counting of the time and the number of times of mastication may be stopped.

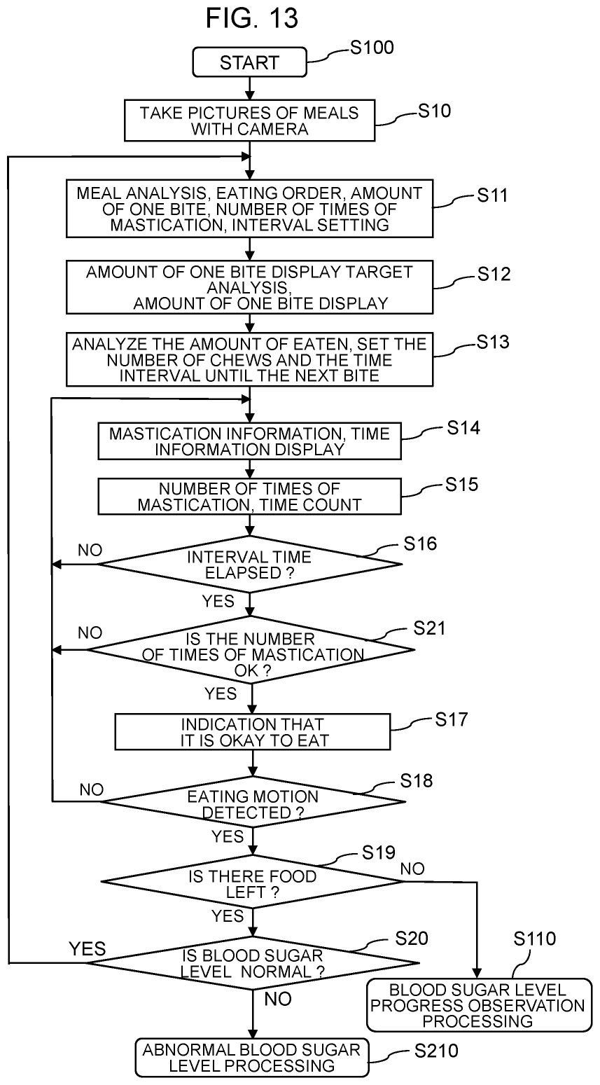

FIG. 13 is an another example of the processing flowchart of the blood sugar level management method in this embodiment. In FIG. 13, the same steps as in FIG. 12 are marked with the same symbols and their descriptions are omitted. The difference between FIG. 13 and FIG. 12 is that step S21 is added between steps S16 and S17.

In FIG. 13, if it is determined in step S16 that the standard interval time until the next bite has elapsed, the process proceeds to step S21 to determine whether the standard number of times of mastication has been cleared, and if so, it moves to step S17 to display a message indicating that it is OK to eat ((see FIG. 4E). That is, FIG. 13 is an example of determining whether to display that it is OK to eat based on both the elapsed time and the number of times of mastication. As a result, by making the standard number of times of mastication, it is possible to easily obtain a feeling of satiety and prevent overeating, and furthermore, it is possible to suppress the increase in the blood sugar level by providing the time interval until the next bite.

In addition, as in FIG. 12, even if both the elapsed time interval and the number of times of mastication are OK, the display and counting of the time and the number of times of mastication until the user enters the next bite motion, but the display and counting of the time and the number of times of mastication may be stopped.

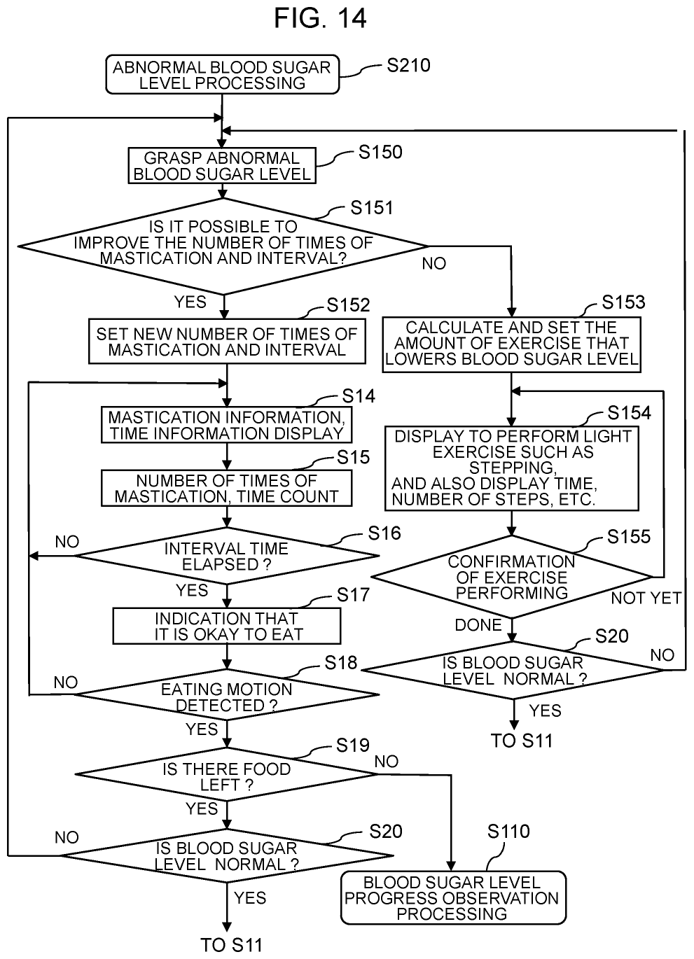

FIG. 14 is a processing flowchart of abnormal blood sugar level processing in this embodiment. In FIG. 14, in the abnormal blood sugar level processing S210, first, in step S150, the extent of the abnormal blood sugar level is grasped. Next, in step S151, it is determined whether improvement is possible by changing the number of times of mastication and the time interval until the next bite grasped in step S150.

If the result of step S151 is YES, the process moves to step S152 to set the new number of times of mastication and time interval (S152). After that, the processing is performed along the processing flow of S14 to S20 and S110 described in FIG. 12.

If the result of step S151 is NO, the process moves to step S153 to select a light exercise, such as stepping, that lowers blood sugar level, and to calculate and set the amount of exercise. Then, in step S154, the set exercise (for example, stepping) and the amount of exercise (for example, 20 footsteps) are displayed on the display 10.

Then, in step S155, various sensors 5 are used to determine whether or not the displayed exercise has been performed. If the result of step S155 is NO, the process returns to step S154 to remind the user to perform the exercise. If the result of step S155 is YES, the process moves to S20 described in FIG. 12.

Although FIG. 14 is based on the processing flowchart of the blood sugar level management method in FIG. 12, it can be based on the processing flowchart of the blood sugar level management method in FIG. 13, in which case step S21 in FIG. 13 should be added between steps S16 and S17.

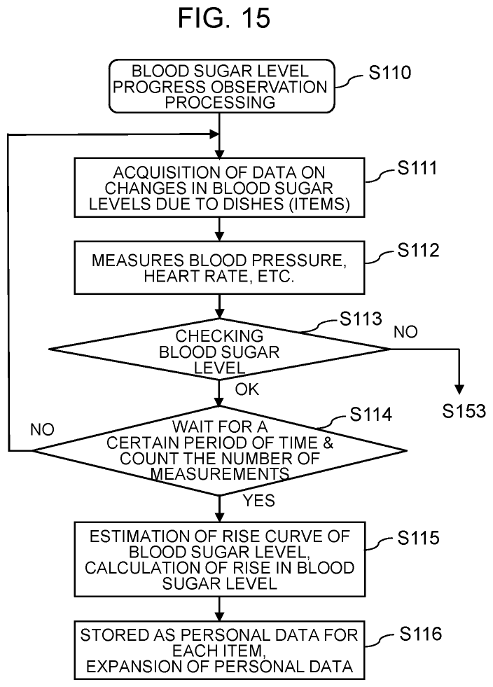

FIG. 15 is a processing flowchart of blood sugar level progress observation processing in this embodiment. In FIG. 15, the blood sugar level progress observation processing S110, first, in step S111, the blood sugar level is measured by the blood sugar level sensor 56 of the HMD 100. Then, in step S112, blood pressure, heart rate, etc. are measured.

In step S113, check whether the blood sugar level measured in step S111 is within the normal range. If the result in step S113 is NO (abnormal), move to step S153 of the abnormal blood sugar level processing in FIG. 14. If the result in step S113 is OK (normal), the process moves to step S114 to wait for a predetermined time (for example, 10 minutes) after the measurement in step S111 and to check whether the number of measurements (for example, 3 times) has been reached.

If the result in step S114 is NO, the process returns to step S111. If the result in step S114 is YES, the process proceeds to step S115 to estimate the rising curve of blood sugar level from the multiple acquired data in steps S111 and S112, and calculate the value at which blood sugar level rises per mouthful (amount of one mouthful).

Then, in step S116, the initial setting data is replenished and updated by storing the dish items (including image data) obtained in FIG. 12 and FIG. 13, the estimated amount of carbohydrates, etc., the amount of one bite, the number of times of mastication, and the interval time until the next bite, along with the identification name, in personal data along with the blood sugar elevation value.

In this way, by accumulating data and updating the initial setting data in step S116, the correlation between the tendency of blood sugar level to rise and the relationship with the dishes can be effectively ascertained and the accuracy of the eating method to keep the blood sugar level rise within a certain range can be increased, since the same dish is usually served at a certain frequency for meals at home.

As described above, according to the present embodiment, a HMD having a non-invasive wearable blood glucose level sensor or the like is used to present specific eating methods in real time when eating, such as displaying an instruction on the number of times of mastication for controlling eating time to avoid eating too fast, and recommending the order in which dish is eaten. By eating in accordance with the presented method, the user can control the blood sugar level to prevent a sudden rise in blood sugar level and keep it within a certain range, which is effective in preventing diabetes and realizing an effective eating method for weight loss. Therefore, it is possible to provide a HMD and blood sugar level management method employed in same that can provide optimal dietary guidance for keeping blood sugar level within a certain range.

### Second Embodiment

People who have developed diabetes or who are on the verge of developing diabetes are often restricted in how they eat (the number of times of mastication, etc.) and in what they eat. Therefore, it can be assumed that they are sometimes hesitant to eat at restaurants, etc., which are known as gourmet restaurants. In this embodiment, a method of realizing a meal at a restaurant or the like while satisfying dietary restrictions will be described.

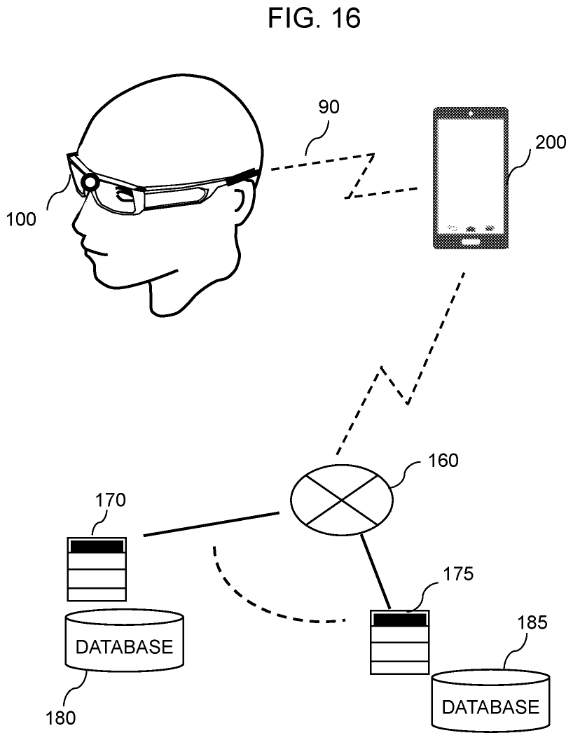

FIG. 16 is a system configuration diagram for realizing the blood sugar level management method in this embodiment. In FIG. 16, the user of this system wears the HMD 100 at a restaurant, for example. The HMD 100 is linked to a mobile terminal 200 via data communication 90, and the mobile terminal 200 can access servers 170 to 175 on the restaurant side via communication network (Internet) 160. The servers 170 to 175 also have databases 180 to 185.

In order to provide eating method that keeps the range of elevated blood sugar level within a certain range at restaurants, it is necessary to know how much carbohydrate (an ingredient that raises blood sugar level) and how much fiber (an ingredient that reduces the rise in blood sugar level) are in the meal. If the amount of carbohydrates and fiber are known, it will be possible to effectively suppress the rise in blood sugar level. Therefore, in order to grasp the amounts of carbohydrates, fiber, etc. contained in menus (dishes) at restaurants almost accurately, the databases 180 to 185 of the amounts of carbohydrates, fibers, etc. for each menu are maintained on the servers 170 to 175 on the restaurant side.

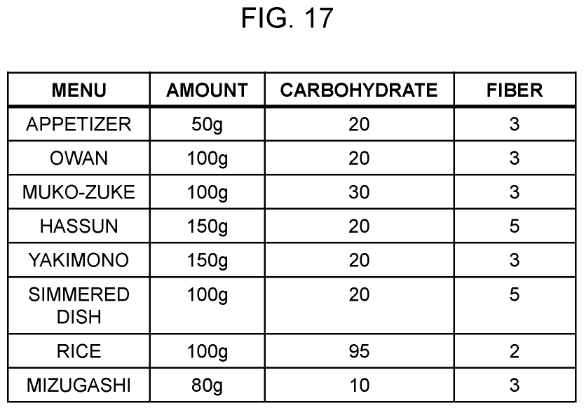

FIG. 17 is an example of a database of carbohydrate and fiber amounts for each menu. As shown in FIG. 17, the database for a given restaurant holds the amount of dish, the amount of carbohydrates, and the amount of fiber for each menu.

The HMD 100 connects to the databases 180 to 185 of the amount of carbohydrates, fiber, etc. in the menu offered by the restaurant via the mobile terminal 200. The HMD 100 uses the information in the databases 180 to 185 to determine the number of times of mastication for each sequentially served dish and the interval time until the next bite, and displays the instructions on display 10.

If the blood sugar level is estimated to rise above a certain range (In the case of NO in S20 in FIG. 12), which corresponds to the abnormal blood sugar level processing S210, along with executing the abnormal blood sugar level processing of FIG. 14, it is also possible to send a request to the servers 170 to 175 on the restaurant side through the mobile terminal 200 to reduce the amount of meal and provide more effective abnormal blood sugar level processing.

As described above, according to the present embodiment, it is possible to realize a meal at a restaurant or the like while meeting dietary restrictions. In addition, the restaurant side can also provide a service to control the rise in blood sugar level and enjoy a pleasant meal.

### Third Embodiment

In embodiments 1 and 2, the method for coping with the case where the blood sugar level rises during a meal has been explained, but in this embodiment, the method for coping with hypoglycemia will be explained.

It is said that hypoglycemia is likely to occur due to a small amount eaten, hard work or exercise on an empty stomach.

It is also said that if too much intake of sugary or calorie-rich foods such as snacks, carbonated drinks, juice drinks, etc. becomes the norm, the pancreas becomes tired and overreacts, resulting in abnormal insulin secretion, which in turn leads to hypoglycemia. Hypoglycemia causes lethargy, distraction, headache, nausea, and in severe cases, coma, which can be very dangerous.

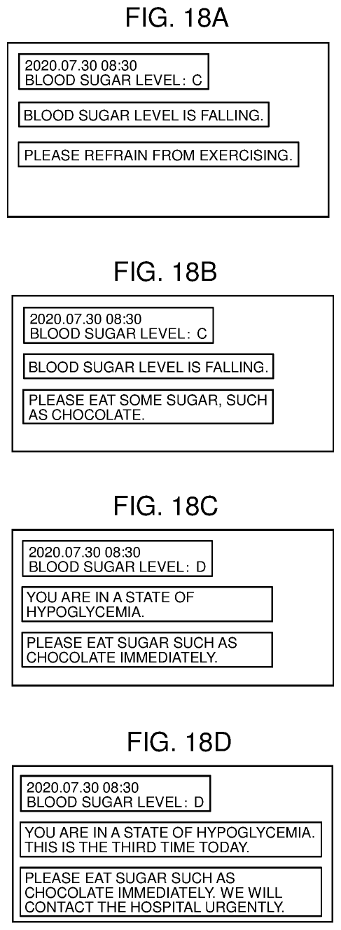

In this embodiment, the HMD 100 is worn at all times, not just during meals, and is connected to the mobile terminal 200 via data communication. In this situation, the measurement of blood sugar level is started. Then, when the blood sugar level is below a certain range, an instruction as shown in FIG. 18A to FIG. 18D are displayed on the display 10 of the HMD 100 in AR.

FIG. 18A shows the display when it can be inferred from the data of various sensors of the HMD 100 that the user is exercising or doing hard labor, and some instructions, such as “Blood sugar level is falling. Please refrain from exercising.”, are displayed in AR.

When the drop in the blood sugar level falls below a certain value, as shown in FIG. 18Bb, some instructions, such as “Blood sugar is falling. Please eat some sugar, such as chocolate.” are displayed.

When the blood sugar level continues to drop or drops significantly below a certain value, as shown in FIG. 18C, some instructions, such as “You are in a state of hypoglycemia. Please eat sugar such as chocolate immediately.” are displayed.

In addition, by continuously measuring the blood sugar level, it is possible to grasp the occurrence of hypoglycemia, and as shown in FIG. 18D, some instructions, such as “You are in a state of hypoglycemia. This is the third time today. Please eat sugar such as chocolate immediately. We will contact the hospital urgently.” are displayed.

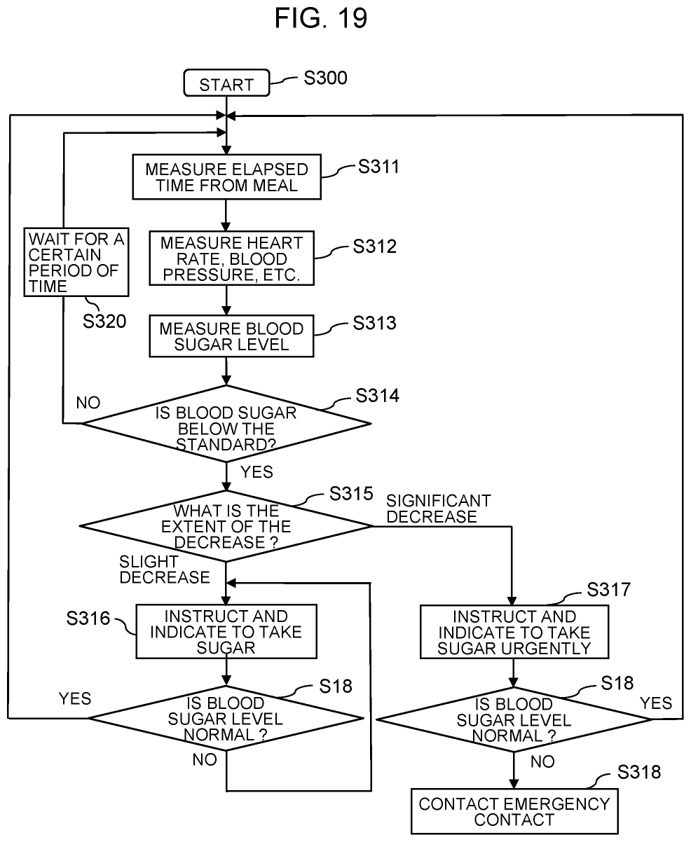

FIG. 19 is a processing flowchart of the process corresponding to hypoglycemia in this embodiment. In FIG. 19, first, at step S300, the user wears the HMD 100 and selects the start of the response to hypoglycemia program with button switch 91. Next, in step S311, the time elapsed since the meal is taken is measured. Then, in step S312, the various sensors of the HMD 100 are used to measure the heart rate, blood pressure, body temperature, etc. to determine whether the user is exercising or not. Also, in step S313, blood sugar level are measured.

Then, in step S314, it is determined whether the blood sugar level corresponds to hypoglycemia from the measurement data of S311 to S313, and each data and instruction content are stored in the flash memory as personal data.

If the result of step S314 is YES (hypoglycemia), the process proceeds to step S315 to determine how much the blood sugar has decreased relative to the reference blood sugar. If the result of step S314 is NO (normal), the process waits for a certain period of time in step S320, and when the waiting time expires, returns to step S311 to repeat the measurement.

If the result of step S315 is a slight decrease, the process proceeds to step S316 to display information such as taking sugar and discontinuing exercise. Then, in step S18, the blood sugar level is measured to confirm the effect of step S316, and it is determined whether or not the blood sugar level is within the normal range. If the result of step S18 is YES (normal), return to step S311. If the result of step S18 is NO (low blood sugar level), return to step S316 and repeat the instructions.

If the result of step S315 is a significant decrease, the process proceeds to step S317 to indicate that sugar should be taken immediately and exercise should be discontinued. Then, in step S18, the blood sugar level is measured to confirm the effect of step S317, and it is determined whether or not the blood sugar level is within the normal range (S18). If the result of step S18 is YES (normal), return to step S311. If the result of step S18 is NO (low blood sugar level), the process proceeds to step S318, and an emergency contact (or 119th ambulance) is contacted using the voice data stored in the HMD 100 together with the location information (S318).

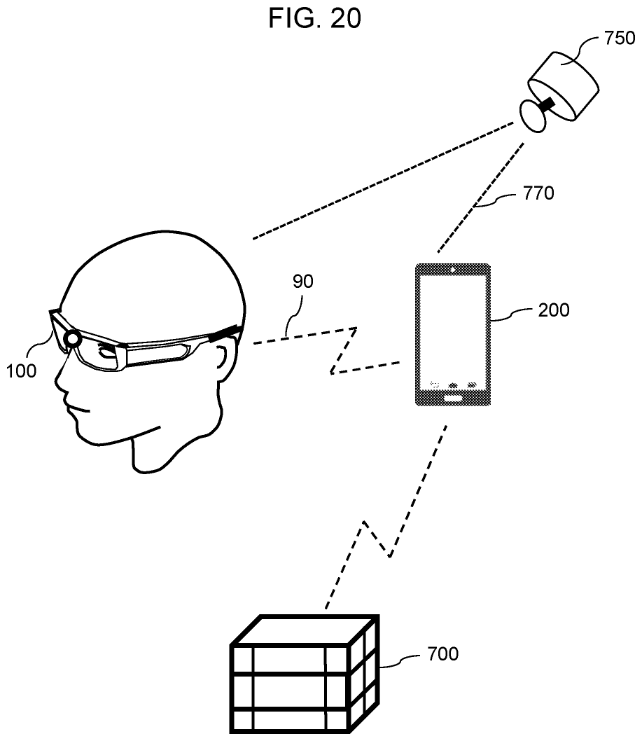

As shown in FIG. 20, the configuration for emergency contact is such that position data of the person wearing the HMD 100 is generated by the mobile terminal 200 or the HMD 100 based on GPS (Global Positioning System) positioning data 770 from the artificial satellite 750, and Physical information such as blood sugar level data, blood pressure, heart rate, etc. is communicated from the mobile terminal 200 to the family doctor 700 together with the location data.

As described above, according to the present embodiment, it is possible to provide a HMD and blood sugar level management method employed in same that capable of coping with hypoglycemia by utilizing a HMD having a non-invasive wearable blood sugar level sensor or the like.

### Fourth Embodiment

In embodiments 1 and 2, instructions were displayed on the display of the HMD 100 to increase the number of times of mastication and to take the interval time until the next bite to keep blood sugar level within a certain range by allowing sufficient time to eat and decreasing the amount to eat. In contrast, in this embodiment, in addition to this method, a method of deceiving the brain (providing false information to the brain), for example, the amount to eat is shown to be larger than the actual amount, the amount of one bite is shown to be larger than the actual amount, etc., to make the person feel as if he/she has consumed a large meal and reduce the amount to eat.

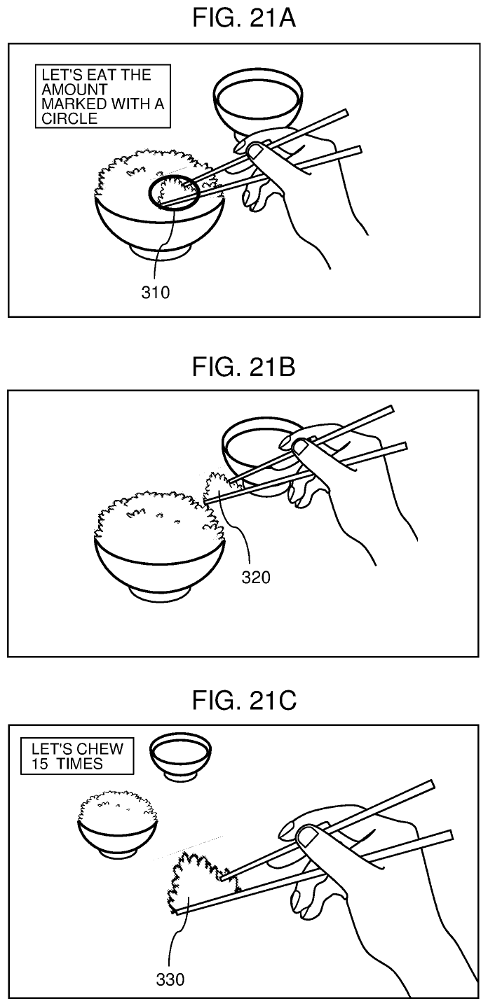

FIG. 21A to FIG. 21C are examples of the display of the HMD 100 in this embodiment. FIG. 21A to FIG. 21C are displays of a case in which the user is about to eat rice with chopsticks during a meal. A spoon may be used instead of chopsticks. In FIG. 21A to FIG. 21C, when taking rice with chopsticks, first, the recommended amount of one bite is displayed in AR as shown in 310 of FIG. 21A, and the user is instructed to catch the recommended amount with chopsticks as shown in 320 of FIG. 21B. Then, as shown in 330 of FIG. 21C, when the food taken with chopsticks is brought to the mouth, a larger amount (virtual one) is displayed in AR instead of the actual amount taken. This makes the amount of rice seem larger, so the amount of rice actually taken is reduced.

As another method, the size of the chopsticks and spoons is reduced in AR (the chopsticks and spoons are replaced with virtual objects) and display them on the display 10. For the wearer of the HMD 100, chopsticks and spoons look small, so they feel that the amount of rice is relatively large, and therefore, the actual amount of rice taken is smaller. Alternatively, instead of making the chopsticks and spoons smaller, the serving bowl of food can be made smaller to make it appear (create the illusion) that the amount of dish is larger.

As described above, according to the present embodiment, it is possible to effectively keep the rise in blood sugar level within a certain range by using virtual objects to reduce the amount to eat and by instructing the user to increase the number of times of mastication and to take the interval time until the next bite.

By creating the virtual object in advance and storing it in the flash memory of the HMD 100, it is possible to reduce the load of the AR processing.

Although the embodiments have been described above, the above described embodiments have been described in detail in order to explain the present invention for easy understanding, and are not necessarily limited to those having all the described configurations. In addition, it is possible to replace part of the configuration of one embodiment with the configuration of another embodiment, and it is also possible to add the configuration of another embodiment to the configuration of one embodiment. Moreover, it is possible to add, delete, or replace a part of the configuration of each embodiment with another configuration.

### REFERENCE SIGNS LIST

4 Control circuit (control device)
5 Sensor
20 Camera
53 Distance sensor
54 Acceleration sensor
55 Gyro sensor
56 Blood sugar level sensor
100 Head mounted display (HMD)
170, 175 Server
180, 185 Database
200 Mobile terminal (Smartphone)
201 Display area
202, 203, 310-330, 410-440, 510, 610-640, 810-860 AR object
700 Family doctor

1. A head mounted display that displays AR objects in a real space, comprising:
a camera that acquires a captured image by capturing an image of the real space;
a blood sugar level sensor that measures the blood sugar level; and
a control device,
wherein the control device determines, from information about a real food obtained from the captured image and information obtained from the blood sugar level sensor, an amount eaten in one bite and a time interval until the next bite, and the amount eaten in one bite and the time interval until the next bite are displayed as the AR objects.

2. The head mounted display according to claim 1, further comprising:
a motion detection sensor that detects vibration and acceleration,
wherein a mastication information is detected by the motion detection sensor, and
the control device determines a recommended number of times of mastication based on the information of real food obtained from the captured image and the measurement information of the blood sugar level sensor, measures a executed number of times of mastication based on the mastication information, and displays a message related to mastication based on the recommended number of times of mastication and the executed number of times of mastication.

3. The head mounted display according to claim 2,
wherein the control device displays a message to make a time interval longer than the time interval until the next bite when the measured value of the blood sugar level sensor exceeds a certain range.

4. The head mounted display according to claim 2,
wherein the control device displays a message to perform an exercise when it is determined that the measured value of the blood sugar level sensor is greater than a certain range.

5. The head mounted display according to claim 4,
wherein the control device determines whether or not the exercise has been performed based on a detection data from the motion detection sensor.

6. A blood sugar level management method using a head mounted display that displays AR objects in real space, comprising:
obtaining a captured image by capturing an image of the real space;
measuring a blood sugar level of a wearer;
determining an amount eaten in one bite and a time interval until the next bite from an real food information obtained from the captured image, and the measured blood sugar level; and
displaying the amount eaten in one bite and the time interval until the next bite as an AR objects.

7. The blood sugar level management method according to claim 6,
wherein detecting a mastication information of the wearer by a motion detection sensor,
determining a recommended number of times of mastication from the real food information obtained from the captured image, and the measured blood sugar level,
measuring a executed number of times of mastication from the mastication information, and
displaying a message related to mastication based on the recommended number of times of mastication and the executed number of times of mastication.

8. The blood sugar level management method according to claim 7,
wherein displaying a message to make a time interval longer than the time interval until the next bite when the measured blood sugar level exceeds a certain range.

9. The blood sugar level management method according to claim 7,
wherein displaying a message to perform an exercise when it is determined that the measured blood sugar level is greater than a certain range.

10. The blood sugar level management method according to claim 9,
wherein determining whether or not the exercise has been performed by the motion detection sensor.
